(19) United States
(12) Patent Application Publication
(10) Pub. No.: US 2021/0386901 A1
(43) Pub. Date: Dec. 16, 2021

(54) Lighting Fixture Having Ultraviolet Disinfection Means

(71) Applicant: OLYMPIA LIGHTING, INC., Northvale, NJ (US)

(72) Inventor: Ram Shalvi, Closter, NJ (US)

(21) Appl. No.: 17/347,950

(22) Filed: Jun. 15, 2021

Related U.S. Application Data
(60) Provisional application No. 63/039,652, filed on Jun. 16, 2020.

(51) Int. Cl. A61L 9/20

(52) U.S. Cl. CPC A61L 9/20; A61L 2209/15; A61L 2209/12; A61L 2209/111

(57) ABSTRACT

The present invention provides a lighting system for disinfection and decontamination of an air stream that is moved by an air circulation means into the interior of the lighting system through an inlet, filtered by an air filtration means, and irradiated by ultraviolet light emitted by ultraviolet light sources, and lastly exhausted via an outlet, and thereby the lighting system disinfects and decontaminates the air and adjacent surfaces. The ultraviolet light is contained within the system, so that a person adjacent to or present within a predetermined distance is not exposed to unsafe ultraviolet radiation. The light sources can be UV, UVC, Near UVA and Visible Light LEDs with a long-lasting rated life. The lighting system may be operated by a smart light switch, wireless communication of a mobile device, a smart home control appliance, and/or built-in motion sensors and software.

### CROSS-REFERENCE TO RELATED APPLICATIONS

This application claims the benefits of U.S. Ser. No. 63/039,652, filed Jun. 16, 2020. The entire contents and disclosures of the prior application are incorporated herein by reference into this application.

Throughout this application, various references are referred to and disclosures of these publications in their entireties are hereby incorporated by reference into this application to more fully describe the state of the art to which this invention pertains.

### FIELD OF THE INVENTION

The present invention relates to lighting systems that include disinfection and/or decontamination by ultraviolet (UV) radiation.

### BACKGROUND OF THE INVENTION

Ultraviolet radiation can be utilized for germicidal disinfection and decontamination of air and surfaces. UV radiation is the electromagnetic radiation that falls in the region of spectrum between visible light and x-rays. UV radiation is invisible to the human eye and includes wavelength in the spectral range of 100 to 400 nanometers (nm). This spectral range can be subdivided into four regions: vacuum UV rays with wavelength in the range of 100 to 200 nm, UVC rays with wavelength range of 200 to 280 nm, UVB rays with wavelength range 280 to 315 nm, and UVA rays with wavelength range 315 nm to 400 nm. Because of the spectral sensitivity of DNA and RNA in bacteria and viruses, only the UVC region demonstrates significant germicidal properties. According to the 2006 U.S. EPA UV Disinfection Guidance Manual (1), recommended UVC exposure dosage, which is measured as the product of UVC light intensity multiplied by exposure time, should be at least 2,500 μW·s/cm² and up to 8,000 μW·s/cm² for effectively killing 90% of most bacteria and viruses.

Conventional devices using UV radiation for disinfection and/or decontamination, such as low-pressure UV lights and/or UV lamps, may have present problems with safety and effectiveness. Extensive and prolonged exposure to UV radiation may be associated with occurrence of skin cancers and may also cause eyesight and eye damage. The effectiveness index of a conventional UV lamp is typically around 80%, which does not put the UVC region into most effective use. The present invention solves foregoing issues with innovative designs and optimal spectral tuning.

### SUMMARY OF THE INVENTION

The present invention provides a lighting system for disinfection of an air stream passing therethrough. The lighting system comprises a front cover assembly comprising detachably embedded light transmissive members, a detachable inlet for receiving air, and a detachable outlet for exhausting air, wherein a stream of air is passable from the inlet to the outlet; a casing frame comprising a first set of mounting openings and a second set of mounting openings; a plurality of ultraviolet LED light sources mounted on the first set of mounting openings and configured to emit ultraviolet light with adjustable wavelengths to irradiate air passing through the interior of the lighting system; a plurality of visible light sources mounted on the second set of mounting openings and configured to emit visible light with adjustable luminous intensity and/or color temperature, wherein the visible light transmits through the light transmissive members and propagates outside the lighting system.

The invention further provides an air circulation means detachably attached to the first set of mounting openings and configured to continuously move a stream of air (a) through the inlet, from outside into the interior of the lighting system, (b) within the lighting system, from the inlet towards the outlet, and (c) through the outlet, from the interior to outside the lighting system; and an air filtration means attached to the air circulation means, facing the inlet and/or the outlet on the front cover assembly, and configured to remove particulates from a stream of air, wherein the front cover assembly is detachably and securely attached to the casing frame, such that the light transmissive members and the second set of mounting openings are aligned.

The front cover assembly and the casing frame jointly form the exterior of the lighting system as an enclosure covering the ultraviolet light sources, the visible light sources, the air circulation means and the air filtration means, and protecting persons near the lighting system from exposure to the ultraviolet light emitted by the ultraviolet light sources; wherein operating conditions and parameters of the plurality of ultraviolet light sources and the plurality of visible light sources may be adjusted independently; and, when the lighting system is operating, a stream of air moved by the air circulation means enters the interior of the lighting system, is filtered by the air filtration means and irradiated by the ultraviolet light sources, and exits the lighting system through the outlet, thereby being disinfected and decontaminated by the lighting system.

The lighting system of the present invention may further comprise UVC sensors inside the front cover assembly and motion sensors, such as High Frequency Doppler (HFD) sensors.

In one embodiment, the UV radiation emitted by the ultraviolet LED light sources has a wavelength range of about 200 nm to about 400 nm. In one embodiment, the UV radiation emitted by the ultraviolet LED light sources is in the wavelength range of about 240 nm to about 290 nm. In another embodiment, the UV radiation emitted by the ultraviolet LED light sources has peak wavelength range of about 260 nm to about 270 nm. In one embodiment, the UV radiation emitted by the ultraviolet LED light sources breaks down and inactivates infectious organisms such as bacteria, viruses, and other pathogens, and thereby disinfects ambient air and adjacent surfaces.

### BRIEF DESCRIPTION OF THE FIGURES

FIG. 1 is a perspective view of one embodiment of the lighting system in accordance with the present invention.

FIG. 2 is the perspective view of the embodiment illustrated in FIG. 1 with the front cover assembly removed.

FIG. 3 is an exploded view of the embodiment illustrated in FIG. 1.

FIG. 4 is a side view of the embodiment illustrated in FIG. 1, with the side of the casing frame removed.

FIG. 5 is an enlarged view of the inlet of the embodiment illustrated in FIG. 1.

FIG. 6 is an enlarged view of the inlet and the LED array of the embodiment illustrated in FIG. 1, with the cover portion partially broken away.

FIG. 7 is a perspective view of one embodiment of the lighting system in accordance with the present invention.

### DETAILED DESCRIPTION OF THE INVENTION

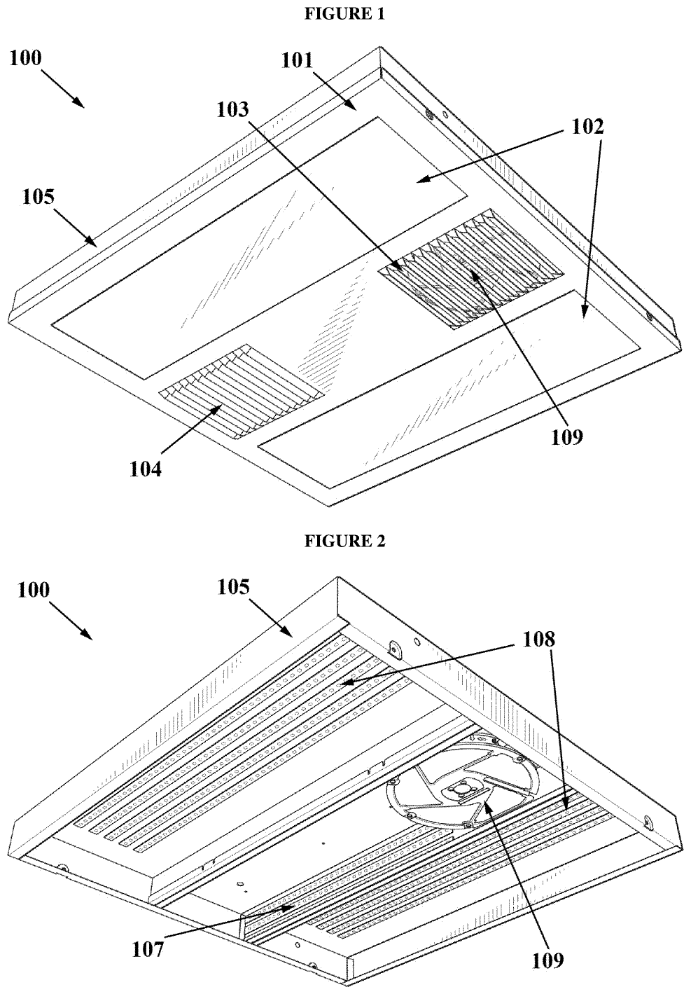

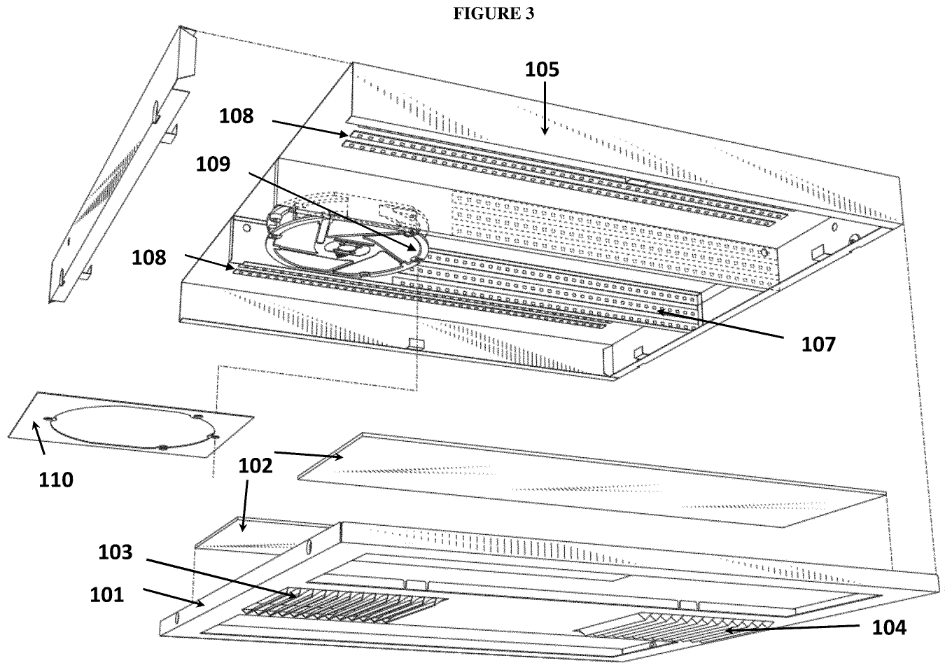

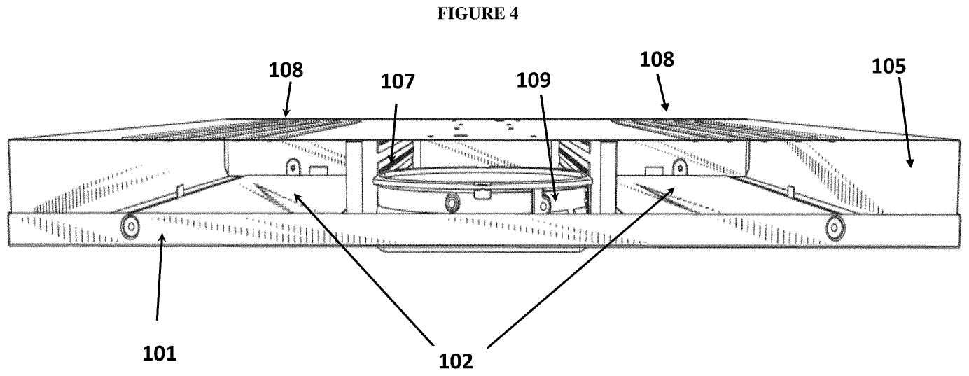

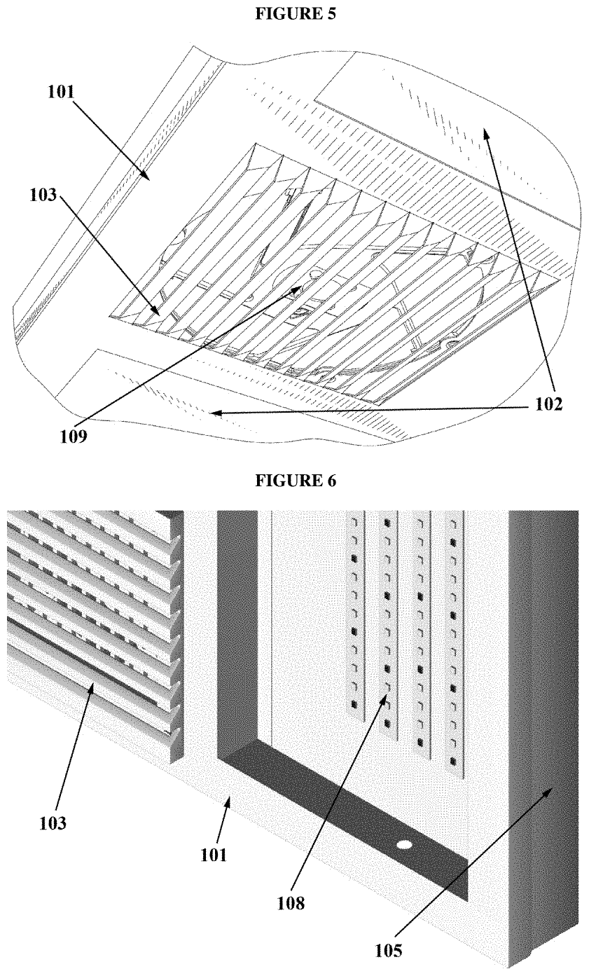

Referring to the drawings, FIG. 1 illustrates an embodiment of the lighting system (100) for disinfection and decontamination of an air stream of the present invention. The lighting system (100) comprises a front cover assembly (101) comprising detachably embedded light transmissive members (102), a detachable inlet (103) for receiving air, and a detachable outlet (104) for exhausting air, wherein a stream of air is passable from the inlet (103) to the outlet (104). The invention further provides a casing frame (105) comprising a first set of mounting openings (106) and a second set of mounting openings (106); a plurality of ultraviolet light sources (107) detachably mounted on the first set of mounting openings (106) and configured to emit ultraviolet light with adjustable wavelengths to irradiate air in the interior of the lighting system (100), as illustrated in FIG. 3. The lighting system (100) has a plurality of visible light sources (108) detachably mounted on the second set of mounting openings (106) and configured to emit visible light with adjustable luminous intensity and/or color temperature, wherein the visible light transmits through the light transmissive members (102) and propagates outside the lighting system (100); an air circulation means (109) detachably attached to the first set of mounting openings (106) and configured to continuously move a stream of air (a) through the inlet (103), from outside into the interior of the lighting system (100), (b) within the lighting system (100), from the inlet (103) towards the outlet (104), and (c) through the outlet (104), from the interior to outside the lighting system (100); and an air filtration means (110) adjacent to the air circulation means (109), facing the inlet (103) and/or the outlet (104) on the front cover assembly (101), and configured to remove particulates from a stream of air, wherein the front cover assembly (101) is detachably and securely attached to the casing frame (105), such that the light transmissive members (102) and the second set of mounting openings (106) are aligned. In one embodiment, the front cover assembly (101) and the casing frame (105) jointly form the exterior of the lighting system (100) as an enclosure protecting the ultraviolet light sources (107), the visible light sources (108), the air circulation means (109) and the air filtration means (110), and protecting a person adjacent to or present within a predetermined distance of the lighting system (100) from exposure to the ultraviolet light emitted by the ultraviolet light sources (107); operating conditions and parameters of the plurality of ultraviolet light sources (107) and the plurality of visible light sources (108) may be adjusted independently; and, when the lighting system (100) is operating, a stream of air moved by the air circulation means (109) enters the interior of the lighting system (100), is filtered by the air filtration means (110) and irradiated by the ultraviolet light sources (107), and exits the lighting system (100) via the outlet (104), thereby being disinfected and decontaminated by the lighting system (100).

The lighting system (100) of the present invention may further comprise UVC sensors inside the front cover assembly (101) and motion sensors such as High Frequency Doppler (HFD) sensors.

The following terms shall be used to describe the present invention. In the absence of a specific definition set forth herein, the terms used to describe the present invention shall be given their common meaning as understood by those of ordinary skill in the art.

The front cover assembly (101) is preferably rigid. It can be in any shape that fits the shape of the casing frame (105) and can be detachably attached to the casing frame (105). When assembled, the front cover assembly (101) and the casing frame (105) form an enclosure to provide protection for parts and components mounted on the casing frame (105) inside the lighting device (100) and to prevent UV rays from emanating outside the lighting system (100). The front cover assembly (101) comprises embedded light transmissive members (102), the inlet (103) and the outlet (104) allowing a passable air stream.

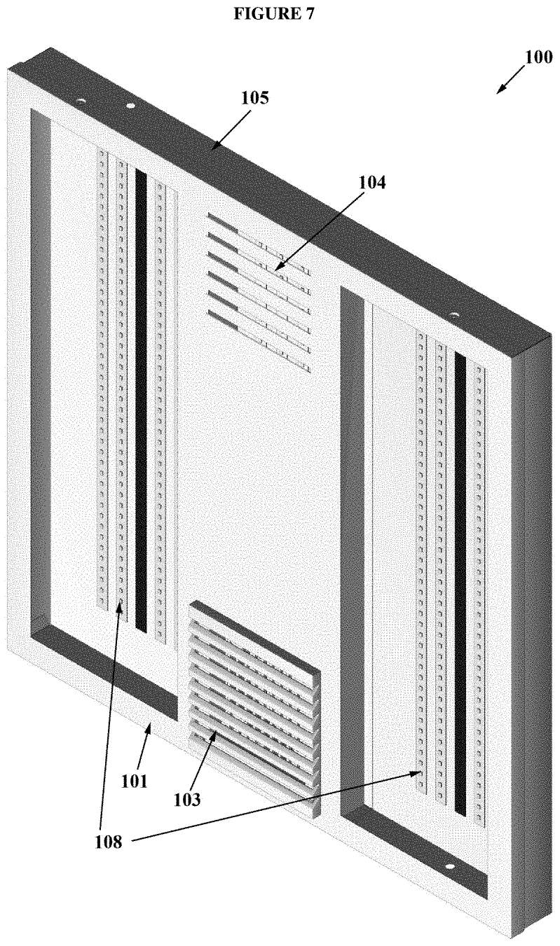

The light transmissive members (102) are panels or lenses made of glass or plastic resins (also termed as plastic organic glass) such as Plexiglass, Polycarbonate (PC), acrylic (PMMA) or Polystyrene (PS). As illustrated in FIG. 6, in one embodiment, the visible light sources (108) comprise strips of interspersed Visible Light LEDs (grey squares; for emitting visible light) and Near UVA LEDs (dark squares; for emitting UVA rays at approximately 405 nm and blue light). As illustrated in FIG. 7, the visible light sources (108) comprise strips of Visible Light LEDs (grey strips; for emitting visible light) and strips of Near UVA LEDs (dark strips; for emitting UVA rays at approximately 405 nm and blue light). The light transmissive members (102) can be transparent, translucent, semi-transparent, or semi-opaque. The light transmissive members (102) are detachably embedded in the front cover assembly (101), and choice of suitable glass or plastic materials depends on actual needs in practice and use. The light transmissive members (102) serve as windows for light emitted by the visible light sources (108) to transmit outside the lighting system (100).

The inlet (103) is a set of slit openings (106) detachably embedded in the front cover assembly (101) that allows air to enter from outside into the interior of the lighting system (100). The slit width and separation can vary, depending on actual needs in practice and use.

The outlet (104) is a set of slit openings (106) on the front cover assembly (101) and allows air to exhaust from the interior to outside the lighting system (100). The slit width and separation can vary, depending on actual needs in practice and use.

The casing frame (105) preferably has a rigid main frame onto which other internal components of the lighting system (100) can be mounted. The casing frame (105) may take any shape (such as square, rectangular, circular, elliptical, ring, etc.), and choice of the shape of the casing frame (105) depends on actual needs in practice and use. As illustrated in FIG. 3, in one embodiment, the casing frame (105) has three mounting openings (only two are shown in the figure due to obstruction). A front cover assembly (101) that matches the shape of the casing frame (105) can be attached thereto, so that they jointly form a protective enclosure for internal parts/components to prevent persons near the lighting system (100) from exposure to UV radiation. The casing frame (105) comprises two sets of openings (106) for mounting the light sources, with one set for mounting the ultraviolet light sources (107), and the other set for mounting the visible light sources (108). The casing frame (105) may fit into a suspended ceiling, or any ceiling commonly available in a house, an office building, or other architecture.

In one embodiment, the casing frame (105), and thus the lighting system (100), is a slim cuboid in shape with 1 to 6 inches in thickness and may have length and width as listed in TABLE 1 (imperial units for North America) or TABLE 2 (metric units for Europe, Asia, and other areas of the world where metric units are used in practice). In one embodiment, the casing frame (105) has the same structure, construction, design and features, regardless of its actual dimensions.

TABLE 1
Length and Width of a Cuboid-shaped Casing Frame

| Length of the Casing | Width of the Casing |
|---|---|
| Frame (in ft.) | Frame (in ft.) |
| 2 | 2 |
| 2 | 4 |
| 1 | 4 |

TABLE 2
Length and Width of a Cuboid-shaped Casing Frame

| Length of the Casing | Width of the Casing |
|---|---|
| Frame (in cm) | Frame (in cm) |
| 60 | 60 |
| 60 | 120 |
| 30 | 120 |

In one embodiment, the dimensions of the casing frame (105), and thus those of the lighting system (100), can be manufactured and customized by customers. In one embodiment, the customized dimensions of the casing frame (105), and thus those of the lighting system (100), are slightly adjusted by no more than ±5% before manufacturing.

The mounting openings (106) are openings on the casing frame (105) that allow light sources to be mounted thereto. The mounting openings (106) serve as sockets and provide a mounting assembly for easy installation and replacement of the light sources.

The ultraviolet light sources (107), such as ultraviolet light-emitting diodes (UV LEDs) including LEDs for emitting UVC rays (UVC LEDs), are chosen to emit desired optical wavelengths in the ultraviolet spectrum and are mounted on a first set of the mounting openings (106) on the casing frame (105). In this disclosure, terms such as “ultraviolet light sources”, “UV light sources”, and “UV lightsources”, are used interchangeably. In one embodiment, the light sources comprise LED. As illustrated in FIG. 2, in one embodiment, the ultraviolet light sources (107) comprise strips of UV LEDs. The ultraviolet light sources (107) are enclosed inside the lighting system (100) by covering the casing frame (105) with the front cover assembly (101). The ultraviolet light sources (107) are arranged in a predetermined pattern (e.g., strips) that is desired in practice and use, usually with a fixed spacing for uniform illumination. When powered on, the ultraviolet light sources (107) emit light that projects outwards, irradiates the interior of the lighting system (100), and disinfects and/or decontaminates air and surrounding area.

In one embodiment, the ultraviolet light sources (107) are UV LEDs that have a rated life of up to 30,000 to 50,000 hours (measured as the time of use after which the LEDs' optical output fades to 70% of the original value), which is about 2.5 to 5 times superior to that of typical quartz tubes. In comparison, the rated life of quartz tubes is typically under about 12,000 hours. In one embodiment, the lifetime or longevity of the LED ultraviolet light sources (107) can be increased by using a large number of such LEDs operating at an electrical power lower than the nominal power output, so as to reduce their produced heat. In one embodiment, the lifetime or longevity of the ultraviolet light sources (107) is increased to 200% of their rated life, by operating at an electrical power equal to 50% of their nominal power.

In one embodiment, the lighting system (100) comprises a UVC sensor inside the front cover assembly (101), for detecting the intensity of UVC rays emitted by the ultraviolet LED light sources (107) in the interior of the lighting system (100). In one embodiment, based on sensory data received from the UVC sensor, the lighting system (100) automatically modulates the electric power supplied to the ultraviolet light sources (107) to maintain a constant level of UVC intensity in the interior of the lighting system (100). In one embodiment, the electrical power supplied to brand-new ultraviolet light sources (107) is 70% of the nominal power supply of the ultraviolet light sources (107), and, after use for a period time and when the UVC sensor detects a 10% reduction of the intensity of UVC rays in the interior of the lighting system (100), the lighting system (100) automatically modulates the electrical power supplied to the ultraviolet light sources (107) to 80% of their nominal power supply. In one embodiment, by modulating the electrical power supplied to the ultraviolet light sources (107), the longevity of the ultraviolet light sources (107) can be increased, and the UVC intensity is maintained at a constant level during the use of the lighting system (100).

In one embodiment, the ultraviolet light sources (107) have peak wavelengths in the range of approximately 265 nm for optimal disinfection and decontamination. In one embodiment, the ultraviolet light sources (e.g., UV and/or UVC LEDs) (107) have peak wavelengths in the range of 260 to 270 nm and total optical power output of at least 60 to 80 mW when operating at 500 mA.

The driver, for example LED driver, is an integrated circuit built in the lighting system (100). The driver receives electric power from an outlet, and it is configured to convert and modulate electric power the ultraviolet light sources (107), the visible light sources (108), and the air circulation means (109). The LED driver also supplies electric power to motion detection sensors and UVC sensors, receives sensory signals from these sensors, and sends instructions to modulate operating conditions of the ultraviolet light sources (107), the visible light sources (108), and the air circulation means (109).

Conventional UV disinfection systems rely on UVC radiation at a wavelength of approximately 254 nm and may only achieve Peak Germicidal Disinfection Effectiveness (PGDE) Index of approximately 80%. Compared to those conventional systems, the lighting system (100) of the present invention optimally tunes the wavelength of UVC radiation in the range of 250 to 300 nm to a peak wavelength of approximately 265 nm, so that it can achieve a PGDE Index of almost 100%. In one embodiment, the lighting system (100) achieves a PGDE Index in a range of 85% to 100%. In one embodiment, the lighting system (100) reduces COVID-19 Virus in nearby air by 99.9%.

In one embodiment, the ultraviolet light sources (107) are UV LEDs, and each UV LED may attain a viewing angle of 130 degrees, a forward voltage between 5.0 and 9.0 V when operating at 500 mA, a junction-to-case thermal resistance of 7.0° C./W, and a power dissipation of 4.0 W or no greater than 4.5 W when operating at 500 mA.

In one embodiment, the ultraviolet light sources (107) can tolerate a continuous forward current of 100 to 700 mA or of 500 mA, a reverse voltage of no higher than about 5 V, a case temperature in the range of −10 to 80° C. when operating at 500 mA, a storage temperature of −40 to 100° C., and a junction temperature no higher than 115° C.

In one embodiment, the ultraviolet light sources (107) have preheat or soak temperature between limit temperatures of 150° C. (Tₛₘᵢₙ) and 200° C. (Tₛₘₐₓ), and time for transition between the limit temperatures is 60 to 120 seconds. The ultraviolet light sources (107) have a liquidous temperature (T_{L}) of approximately 217° C., a maximum peak package body temperature (Tᵥ) of 260° C., a maximum ramp-up rate of 3° C./s from, and a time maintained above T_{L} of 60 to 150 seconds (t_{L}). The ultraviolet light sources (107) have a maximum ramp-down rate of 6° C./s from T_{P} to T_{L} and a maximum time of approximately 8 minutes from 25° C. to T_{P}.

In one embodiment, the ultraviolet light sources (107) have a power of 30 to 150 W, a flux of 10 to 10,000 μW/cm² so as to achieve a UVC exposure dosage of at least 2,500 μW·s/cm² in the interior of the lighting system (100) within several seconds to several minutes of use, and an irradiation of 200 to 3,000 mW which is a high UV intensity for fast and effective disinfection and decontamination of an airstream as discussed below.

Compared to conventional UV disinfection systems, the lighting system (100) of the present invention provides safety protection and prevents exposure to UV hazard, such as protecting a user's eye health, by a clever design that contains the UV light sources (107) and UV rays emitted thereby within the enclosure. Exposure to UV radiation can be dangerous and associated with damage to eyesight and incidence of skin cancers, and users of UV systems should be protected from direct exposure of UV radiation. When operating and emitting UVC radiation, the lighting system (100) of the presents invention prevent the UV rays from propagating outside the lighting system (100) to avoid exposure to the UV radiation. Since UV rays, which are invisible to human eyes, remain inside the lighting system (100), safety of use of the lighting system (100) is enhanced.

Ultraviolet rays emitted by the ultraviolet light sources (107) in the present invention irradiate only the interior of the lighting system (100) and do not spread into space outside the lighting system (100), so the lighting system (100) protects against unsafe UV radiation. In one embodiment of the present invention, when the lighting system (100) is operating, UV rays cannot be detected in the outside space in proximity to the lighting system (100). In one embodiment of the present invention, when the lighting system (100) is operating, UV rays detected in the proximity of the lighting system (100) is less than 0.1% of the total optical power of the ultraviolet light sources (107), i.e., less than 0.1 μW/cm², corresponding to an absolute safe level for extensive use by human.

The visible light sources (108) are chosen to emit light only in the visible spectrum (about 380 to 740 nm), and they may mimic the light temperature of daylight, incandescent or fluorescent bulbs, and/or provide a particular color tone desired for a practical application. The visible light LED array can be manufactured in any shape or pattern as desired in practice and use, but in one embodiment, the visible light source is a plurality or array of visible light LEDs. The visible light sources (108) are detachably mounted onto a second set of mounting openings (106), similar to how the ultraviolet light sources (107) are mounted onto the first set of mounting openings (106). The second set of mounting openings (106) are aligned with the light transmissive members (102) on the front cover assembly (101), so that light emitted by the visible light sources (108) is visible outside the lighting system (100) to illuminate the surrounding space. The visible light sources (108) can cooperate with the ultraviolet light sources (107), i.e., the two kinds of light sources are powered on at the same time. Alternatively, the visible light sources (108) can be turned off, while the ultraviolet light sources (107), the air circulation means (109), and the air filtration means (110) may remain operating for disinfection and decontamination of air and adjacent surface during nighttime. Depending on the operator's preference, a user may also turn on the visible light sources (108) alone while keeping the UV light sources (107) off.

In one embodiment, the visible light sources (108) are Visible Light LEDs that emit visible light, including but not limited to visible light rays with color temperature between 3,000 K and 5,000K, as well as near UVA rays at approximately 405 nm and blue light. In one embodiment, the visible light sources (108) comprise both Visible Light LEDs that emit visible light and Near UVA LEDs that emit UVA rays at an approximate wavelength of 405 nm and blue light. UVA rays at a wavelength of approximately 405 nm, as well as the blue light, are visible to the human eye, and their bactericidal effects, i.e., inactivation of bacteria such as Escherichia, Salmonella, Shigella, Listeria, and Mycobacterium species are demonstrated by a previous study (2). In one embodiment, the visible light sources (108) comprise both Visible Light LEDs and Near UVA LEDs, and the Near UVA LEDs are interspersed with the Visible Light LEDs in proportions that are predetermined during manufacturing process. In one embodiment, the visible light sources (108) comprise strips of Near UVA LEDs interspersed with strips of Visible Light LEDs, and the strips are optionally laid in parallel. In one embodiment, the visible light sources (108) comprise strips of LEDs, each strip comprising Near UVA LEDs interspersed with Visible Light LEDs, and the LEDs on each strip are optionally equally spaced.

In one embodiment, electrical power provided to the Near UVA LEDs and the Visible Light LEDs is independently controlled. The Near UVA LEDs and the Visible Light LEDs can be powered on simultaneously, or the Near UVA LEDs can be powered on while the Visible Light LEDs are powered off, and vice versa. In one embodiment, the Visible Light LEDs are powered off at night, when a user or an operator is asleep or, for example, away from office, while the Near UVA LEDs remain powered on for disinfection of air and adjacent surfaces. In one embodiment, the Near UVA LEDs remain powered on during the day for disinfection of air and adjacent surfaces, while the Visible Light LEDs are turned off.

The following operating conditions and/or parameters are all independently adjustable:
(a) luminous intensity (brightness) and the color temperature of the visible light sources (108);
(b) On/Off states of the Visible Light LEDs and the Near UVA LEDs; and
(c) intensity and wavelength of the ultraviolet light sources (107).

The visible light sources (108) support varying color temperature that can be controllably varied between 3,000K and 5,000K. In one embodiment, color temperature of the visible light sources (108) may vary among 3,000K, 3,500 K, 4,000 K and 5,000 K. In one embodiment, color temperature of the visible light sources (108) may vary continuously from 3,000 K to 5,000 K.

A user or operator may manually adjust or switch between different luminous intensities, different color temperatures and/or different On/Off states for the Visible Light LEDs and the Near UVA LEDs as the user or operator prefers. Furthermore, the luminous intensity, color temperature, and the On/Off states may be automatically adjusted based on electronic instructions, such as:
instructions stored in a smart light switch that is programmable by a user or operator, including the user setting a timer for turning the lighting system (100) on or off or for brightening or dimming the visible light sources (108) at certain preset time;
instruction received over wireless communication including Wi-Fi™ and/or Bluetooth® from a smart device such as a smartphone, a tablet computer, or a wearable smart device such as a smartwatch;
instructions received from a smart home control appliance including a smart speaker such as the Amazon Echo, the Google Home, the Apple Homepod, or other smart appliance of similar functions; and
instructions encoded in the software of the lighting system (100), including instructions based on input data such as sensory data from motion sensors, e.g., High Frequency Doppler (HFD) sensors.

In one embodiment, color temperature of the visible light sources (108) is automatically reduced after sunset and automatically increased after sunrise, rendering a warmer color temperature during the night than during the day.

In one embodiment, the lighting system (100) is automatically turned on or off by a timer function enabled by a smart light switch, a smart device, a smart home control appliance, or built-in software at certain time preset by a user.

In one embodiment, the visible light sources (108) are automatically brightened or dimmed by a brightness control function preset by a user and enabled by a smart light switch, a smart device, a smart home control appliance, or built-in software.

In one embodiment, the wavelength of the UV rays emitted by the UV light sources (107) is automatically adjusted by a wavelength control function enabled by a smart light switch, a smart device, a smart home control appliance, or built-in software.

In one embodiment, the Visible Light LEDs are automatically powered off at night, and meanwhile the Near UVA LEDs automatically remain or are powered on to emit blue light and UVA rays at approximately 405 nm by a visible LED control function to assist in decontamination of bacteria and other pathogens on nearby surfaces.

The lighting system (100) of the present invention may further comprise a motion sensor or detector that is normally used in a surveillance apparatus such as a miniaturized security camera. In one embodiment, the lighting system (100) of the present invention comprises a High Frequency Doppler (HFD) sensor. Sensory signal of the motion sensor can be image-based, and the motion sensor can include a camera system that supports normal camera functionality such as capturing and responding to visual images. The sensory input can also be infrared ray-based, and the motion sensor can include a sensor unit with predetermined field of view. The sensory input can further be microwave-based, such as the HFD sensor, to achieve superior detection range and sensitivity.

In one embodiment, the lighting system (100) of the present invention comprises a motion sensor that transmits sensory signals when motion of a human is detected within a detection range, e.g., 3 to 12 meters. The detection range is tunable during manufacturing process and/or field adjustable via a switch or software. With a detection range up to 12 meters, the lighting system (100) can accommodate a large space with a high ceiling, such as large high-rise hall rooms. In one embodiment, the lighting system (100) comprises a motion sensor with a maximum detection range of up to 12 meters. In one embodiment, the lighting system (100) comprises a motion sensor whose detection range can be field adjusted from 10% to 100% of the maximum detection range. In one embodiment, the lighting system (100) comprises a motion sensor whose detection range can be field adjusted to 10%, 30%, 50%, 75% or 100% of the maximum detection range.

In one embodiment, the motion sensor outputs one of two states, i.e., either motion of a human is detected (STATE 1) or no longer detected (STATE 2) after a predetermined time period that is adjustable between 5 seconds and 30 minutes. As a means for preventing false signals due to noises in surrounding environment, the motion sensor has a built-in sensitivity threshold, such that it only determines that a motion is detected (STATE 1), if the detection signal is above the threshold. The motion sensor sends an output of either STATE 1 or STATE 2 to the lighting system (100), which then adjusts operating conditions or parameters of the ultraviolet light sources (107), the visible light source (108), and the air circulation means (109), independently.

In one embodiment, the motion sensor sends an output of STATE 1 as an instruction, when motion of a human is detected, and upon receiving the instruction, the lighting system (100) carries out one or more operations as follows:
(a) power on the Visible Light LEDs of the visible light sources (108);
(b) power on the Near UVA LEDs of the visible light sources (108);
(c) power on the ultraviolet light sources (107); and
(d) increase the intensity of any of above light sources.
   Each of above operations may be carried out by the lighting system (100) independently.

In one embodiment, the motion sensor sends an output of STATE 2 as an instruction, when motion of a human is not detected for a predetermined time period that can be adjusted between 5 seconds and 30 minutes, and upon receiving the instruction, the lighting system (100) carries out one or more operations as follows:
(a) power off the Visible Light LEDs of the visible light sources (108);
(b) power off the Near UVA LEDs of the visible light sources (108);
(c) power off the ultraviolet light sources (107); and
(d) decrease the intensity of any of above light sources.
   Each of above operations may be carried out by the lighting system (100) independently.

In one embodiment, the lighting system (100) automatically powers on the visible light sources (108) upon receiving a STATE 1 instruction, and powers them off upon receiving a STATE 2 instruction.

In one embodiment, upon receiving the STATE 1 or STATE 2 instruction, the lighting system (100) automatically powers on or off the ultraviolet light sources (107) and the visible light sources (108) simultaneously. In one embodiment, the ultraviolet light sources (107) remain powered on, regardless of the STATE 1 and/or STATE 2 instructions received by the lighting system (100). In one embodiment, as a protection for people who are concerned about exposure to UV radiation, the lighting system (100) automatically powers off the ultraviolet light sources (107) upon receiving a STATE 1 instruction, and powers them on upon receiving a STATE 2 instruction.

In one embodiment, the Near UVA LEDs remain powered on, regardless of the STATE 1 and/or STATE 2 instructions received by the lighting system (100). In one embodiment, upon receiving the STATE 1 or STATE 2 instruction, the lighting system (100) automatically powers on the Visible Light LEDs and the Near UVA LEDs simultaneously. In one embodiment, as a protection for people who are concerned with exposure to UV radiation, the lighting system (100) automatically powers off the Near UVA LEDs upon receiving a STATE 1 instruction, and powers them on upon receiving a STATE 2 instruction.

In one embodiment, the motion sensor sends an output of STATE 2 as an instruction, when motion of a human is not detected for a predetermined time period that can be field adjusted between 5 seconds and 30 minutes by a switch and/or software. In one embodiment, the motion sensor sends an output of STATE 2 as instruction, when motion of a human is not detected for a predetermined time period that can be field adjusted to 5 seconds, 30 seconds, 1 minute, 5 minutes, 10 minutes, 20 minutes and 30 minutes by a switch and/or software.

In one embodiment, the control function triggered by receiving either the STATE 1 or STATE 2 instruction is programmable by a user on a smart light switch, a smart device, a smart home control appliance, or built-in software.

The air circulation means (109) moves air to circulate through the inlet (103) and outlet (104) on the front cover assembly (101) and within the interior of the lighting system (100). When operating, the air circulation means (109) generates air pressure that causes air to enter through the inlet (103) on the front cover assembly (101) into the interior of the lighting system (100), pass within the lighting system (100) from the inlet (103) towards the outlet (104), and exits the lighting system (100) through the outlet (104) on the front cover assembly (101). With the circulation, air stream is pulled from the surrounding space and pushed through the ultraviolet light sources (107) for disinfection and decontamination, before it returns and is distributed in the surrounding space. The air circulation means (109) has adjustable operating speed, so that it may run at a low speed, a high speed, or any other speed between the two. A low or comfortably negligible noise is rendered at low speed, and a high throughput of air stream is enabled at high speed, so that the lighting system (100) is suited for use in a large space despite its seemingly small form factor. In one embodiment, the air circulation means (109) has a flow rate of up to 50 to 100 cu. ft. per minute, i.e., 50 to 100 CFM, when operating at the high speed. The air circulation means (109) is detachably assembled onto the casing frame (105), facilitating replacement when needed.

In one embodiment, the motion sensor sends an output of STATE 1 as an instruction, when motion of a human is detected, and upon receiving the instruction, the lighting system (100) automatically sets the air circulation means (109) to operate at the high speed. In one embodiment, the motion sensor sends an output of STATE 2 as an instruction, when motion of a human is not detected for a predetermined time period, and upon receiving the instruction, the lighting system (100) automatically sets the air circulation means (109) to a lower speed such as the low speed, or powers the lighting system (100) off.

In one embodiment, the air circulation means (109) can be a fan (109) that is propelled by a small AC or DC electric motor. The fan (109) may have 4 to 8 blades as shown in FIG. 2. In one embodiment, the air circulation means (109) may comprise two fans, one facing the inlet (103) and the other facing the outlet (104), which operate at the same time for a strengthened air flow.

In one embodiment, the air circulation means (109) allows adjustment of operating power between two speeds, a high speed for high air flow throughput, and a low speed for comfortably negligible noise. In one embodiment, the air circulation means (109) allows adjustment of operating power among three to five speeds, wherein the lowest speed renders comfortably negligible noise, and wherein the lighting system (100) can serve a large space up to 800 sq. ft. at the highest speed. In one embodiment, several lighting systems (100) of the present invention may cooperate at the high speed of the air circulation means (109) at the same time, so as to accommodate an indoor space of any size.

The air filtration means (110) has an identical or similar shape to the air circulation means (109) and is detachably attached thereto for removal of particulates from the circulated air stream. Efficiency of the air filtration means (110) is increased at a higher operating speed of running of the air circulation means (109). The air filtration means (110) is replaceable, and its easy installation also facilitates regular replacement with a certain period for continuing effectiveness of air decontamination.

In one embodiment, the air filtration means (110) is an air filter commonly available in home-use or commercial air purifiers. In one embodiment, the air filtration means (110) is an HEPA air filter that may be replaced or cleaned. In one embodiment, the air filtration means (110) is replaced or cleaned every month. In one embodiment, the air filtration means (110) is replaced or cleaned every three months. In one embodiment, the air filtration means (110) is replaced or cleaned every six months.

In one embodiment, the air filtration means (110) is one air filter attached to one fan facing the inlet (103) on the front cover assembly (101). In one embodiment, the air filtration means (110) is one air filter attached to one fan facing the outlet (104) on the front cover assembly (101). In one embodiment, the air filtration means (110) comprises two air filters, each attached to one of two fans, respectively and facing the inlet (103) and the outlet (104) on the front cover assembly (101), respectively.

Control of the lighting system (100) can be operated by commonly available switches, such as light switches. Furthermore, the lighting system (100) of the present invention may be operated by smart switches.

In one embodiment, the lighting system (100) for disinfection and decontamination of an air stream circulating therein comprises a rigid front cover assembly (101) comprising detachably embedded light transmissive members (102), a removable inlet (103) for receiving air, and a removable outlet (104) for exhausting air, wherein a stream of air is passable from the inlet (103) to the outlet (104); a rigid casing frame (105) comprising a first set of mounting openings (106) and a second set of mounting openings (106); a plurality of ultraviolet light sources (107) detachably mounted on the first set of mounting openings (106) and configured to emit ultraviolet light with adjustable wavelengths to irradiate air in the interior of the lighting system (100); a plurality of visible light sources (108) detachably mounted on the second set of mounting openings (106) and configured to emit visible light with adjustable luminous intensity and/or color temperature, wherein the visible light transmits through the light transmissive members (102) and propagates outside the lighting system (100); an air circulation means (109), such as a fan detachably attached to the first set of mounting openings (106) and configured to continuously move a stream of air (a) through the inlet (103), from outside into the interior of the lighting system (100), (b) within the lighting system (100), from the inlet (103) towards the outlet (104), and (c) through the outlet (104), from the interior to outside the lighting system (100); and an air filtration means (110) attached to the air circulation means (109), facing the inlet (103) and/or the outlet (104) on the front cover assembly (101), and configured to remove particulates from a stream of air, wherein the front cover assembly (101) is detachably and securely attached to the casing frame (105), such that the light transmissive members (102) and the second set of mounting openings (106) are aligned; the front cover assembly (101) and the casing frame (105) jointly form the exterior of the lighting system (100) as an enclosure protecting the ultraviolet light sources (107), the visible light sources (108), the air circulation means (109) and the air filtration means (110), and protecting a person adjacent to or present within a predetermined distance of the lighting system (100) from exposure to the ultraviolet light emitted by the ultraviolet light sources (107); operating conditions and parameters of the plurality of ultraviolet light sources (107) and the plurality of visible light sources (108) may be adjusted independently; and, when the lighting system (100) is operating, a stream of air moved by the air circulation means (109) enters the interior of the lighting system (100), is filtered by the air filtration means (110), irradiated by the ultraviolet light sources (107), and exits the lighting system (100) via the outlet (104), thereby being disinfected and decontaminated by the lighting system (100).

In one embodiment, the light transmissive members (102) are made of glass or plastic that is transparent, translucent, or semi-opaque.

In one embodiment, the length of the casing frame (105) is 2 to 4 ft., the width of the casing frame (105) is 1 to 2 ft., and the size or dimensions of the casing frame (105) is customizable.

In one embodiment, the ultraviolet LED light sources (107) have a rated life of 30,000 to 50,000 hours, as measured by the time of use when the light output of the ultraviolet light sources (107) fades to 70% of the original light output.

In one embodiment, the wavelength of the ultraviolet light is in the range of 200 to 290 nm, and the peak wavelength of the ultraviolet radiation is in the range of 260 nm to 270 nm or at approximately 265 nm.

In one embodiment, the lighting system (100) has a Peak Germicidal Disinfection Effectiveness (PGDE) Index of approximately up to 100%.

In one embodiment, while emitting UVC rays with wavelengths in the range of 200 to 280 nm, the ultraviolet light sources (107) simultaneously emit UVA rays with wavelengths in the range of 390 to 410 nm and blue light, wherein both the UVA rays and the blue light are visible to the human eye.

In one embodiment, the ultraviolet light sources (107) are configured to emit UVA rays with wavelengths in the range of 390 to 410 nm.

In one embodiment, the ultraviolet light sources (107) and the visible light sources (108) can be turned on and off simultaneously, or each can be turned on while the other is shut off.

In one embodiment, the circulation means (109) comprises a fan facing the inlet (103) on the front cover assembly (101). In one embodiment, the circulation means (109) comprises two fans facing the inlet (103) and the outlet (104) on the front cover assembly (101), respectively.

In one embodiment, the fan speed is adjustable between a low speed for negligible noise level, a high speed for maximum throughput of circulated air, and 1 to 3 intermediate speeds between the low and the high speeds.

In one embodiment, the air filtration means (110) is an air filter or an HEPA filter.

In one embodiment, the front cover assembly (101), the plurality of ultraviolet light sources (107), the plurality of visible light sources (108), the air circulation means (109) and the air filtration means (110) are easily assembled and easily disassembled, thereby facilitating cleaning of parts and replacement of parts that are malfunctioning or reach the end of a life cycle.

In one embodiment, the wavelength of the ultraviolet light and the luminous intensity and color temperature of the visible light are independently adjustable when the lighting system (100) receives one or more instructions including:
(a) an instruction stored in a smart light switch electrically controlling the lighting system (100), wherein such an instruction is programmable by a user;
(b) an instruction received via wireless communication such as Wi-Fi and/or Bluetooth from a transmitting device operated by a user;
(c) an instruction received via a smart home control appliance including a smart speaker wirelessly connected to the lighting system (100) or a smart light switch thereof; and
(d) an instruction generated and encoded by software built into the lighting system (100) for functions thereof.

In one embodiment, the lighting system (100) further comprises motion sensors that can detect motion of a human in the nearby space; wherein, when such motion of a human is detected, software generates instructions based on the sensed motion and other parameters including date and time, environmental temperature, and user information and user input data; and, wherein the generated instructions comprise:
(a) an instruction to change the luminous intensity of the visible light sources (108), including power on, power off, and to increase or decrease the luminous intensity;
(b) an instruction to set the wavelength of the ultraviolet light emitted by the ultraviolet light sources (107) to 405 nm after sunset time; and
(c) an instruction to set the color temperature to a certain value between 3,000 K and 5,000 K.

In one embodiment, the lighting system (100) of the present invention provides a system for disinfection of air and surfaces through UVC irradiation that occurs with a line of sight between the UVC source and the air or the surface.

In one embodiment, the device (100) provides surface and air disinfection for airborne viruses.

In one embodiment, both the plurality of visible light sources (108) for illumination and the plurality of ultraviolet light sources (107) for air disinfection can be powered on simultaneously. Alternatively, the plurality of ultraviolet light sources (107) can stay on, when the plurality of visible light sources (108) for illumination are turned off, and keep disinfecting the air.

In one embodiment, air propelled by the circulation means (109) inside the lighting system (100) of the present invention effectively reduces the operating temperature of the plurality of visible light sources (108) and the plurality of ultraviolet light sources (107).

In one embodiment, the lighting system (100) of the present invention can be instantly turned on or off. In one embodiment, the wavelength of the ultraviolet radiation can be tuned from 250 nm to 285 nm.

In one embodiment, the lighting system (100) achieves a longer lifetime of approximately 30,000 hours, which is superior to Quartz UV Tubes and Lamps that usually have lifetime of around 8,000 hours.

In one embodiment, the lighting system (100) monitors the internal airflow and modulates the intensity of UVC radiation as emitted by the plurality of ultraviolet light sources (107) to effectively deactivate microorganisms such as bacteria, viruses, and mold.

In one embodiment, the lighting system (100) has a built-in detector of internal airflow and automatic adjust the power of the plurality of ultraviolet light sources (107). In one embodiment, the lighting system (100) automatically increases the power of the plurality of ultraviolet light sources (107) when flow rate of the internal airflow is detected to be above a threshold.

In one embodiment, the lighting system (100) has a built-in wide voltage range LED driver operating from 100 to 480 Volts for quick and simple installation.

In one embodiment, the lighting system (100) further comprises magnets or clamps for mounts.

In one embodiment, the lighting system (100) emits visible light with adjustable luminous intensity and/or color temperature, which, in combination with a UVC troffer, allows for installation of the lighting system (100) as a replacement for existing fixture by match the luminous intensity and/or color temperature. In many public buildings, there is a requirement for 25% of the fixtures to be installed on the emergency circuit, and the lighting system (100) is a suitable fixture to be retrofitted, because it comprises adjustable visible light.

In one embodiment, the lighting system (100) comprises a UVC sensor or detector that sends a signal to the LED driver that provides power to the UVC LED light sources to maintain a constant level of UVC intensity in the interior of the lighting system (100).

In one embodiment, the lighting system (100) comprises an airflow sensor or detector that sends a signal to the LED driver that provides power to the UVC LED light sources to adjust the power of the UVC light sources.

In one embodiment, the present invention provides a lighting system (100) for disinfection of an air stream. The lighting system (100) comprises: a front cover assembly (101), which comprises detachably embedded light transmissive members (102), a detachable inlet for receiving air (103), and a detachable outlet for exhausting air (104), wherein a stream of air is passable from the inlet to the outlet; a casing frame (105) including mounting openings (106); a plurality of ultraviolet light sources (107) detachably mounted on the mounting openings (106) and configured to emit ultraviolet light with adjustable wavelengths and adjustable intensity to irradiate air in the interior of the lighting system; a plurality of visible light sources (108) detachably mounted on the mounting openings (106) and configured to emit visible light with adjustable luminous intensity and/or color temperature, wherein the visible light transmits through the light transmissive members and propagates outside the lighting system; and air circulation means (109) detachably attached to the mounting openings (106). In one embodiment, the air circulation means (109) is configured to move a stream of air through the inlet (103), from outside into the interior of the lighting system; within the lighting system, from the inlet (103) towards the outlet (104); and, through the outlet (104), from the interior to outside the lighting system (100).

In one embodiment, the front cover assembly (101) is detachably attached to the casing frame (105), such that the light transmissive members (102) and the mounting openings (106) are aligned. In one embodiment, the front cover assembly (101) and the casing frame (105) jointly form the exterior of the lighting system (100) as an enclosure protecting the ultraviolet light sources (107), the visible light sources (108), and the air circulation means (109).

In one embodiment, when the lighting system (100) is operating, an air stream moved by the air circulation means (109) enters the interior of the lighting system (100), is irradiated by the ultraviolet light sources (107), exits the lighting system (100) via the outlet (104), and thereby is disinfected after passing through the lighting system (100).

In one embodiment, the ultraviolet light sources (107) the lighting system (100) emit UVC rays with wavelength range of 200 to 280 nm.

In one embodiment, the lighting system (100) has a power of 30 to 150 W, a flux of 10 to 10,000 μW/cm2 to achieve an irradiation dosage of at least 2,500 μW·s/cm2 in the interior of the lighting system (100) within several seconds to several minutes of use, and an irradiation of 200 to 3,000 mW for rapid and substantially complete disinfection of the air stream.

In one embodiment, the lighting system (100) further comprises an air filtration means (109) attached to the air circulation means (110) facing the inlet and/or the outlet on the front cover assembly and configured to remove particulates from the air stream.

In one embodiment, the lighting system (100) further comprises a UVC sensor inside the front cover assembly (101) for detecting the intensity of UVC rays emitted by the ultraviolet light sources (107) in the interior of the lighting system (100).

In one embodiment, based on sensory data received from the UVC sensor, a driver of the lighting system (100) automatically modulates the electric power supplied to the ultraviolet light sources (107) to maintain a constant level of UVC intensity in the interior of the lighting system (100).

In one embodiment, 70% of the nominal power supply to the lighting system (100) is provided to brand-new ultraviolet light sources (107) while, when the UVC sensor detects a 10% reduction of the intensity of UVC rays in the interior of the lighting system (100), 80% of their nominal power supply is provided to the ultraviolet light sources (107) after use for a period time.

In one embodiment, the ultraviolet light sources (107) is supplied with modulable electric power and thus have an increased longevity.

In one embodiment, the ultraviolet light sources (107) have a rated life of 30,000 to 50,000 hours, wherein the rated life is a period of time in which the ultraviolet light sources (107) have an optical output of no less than 70% of the nominal power output.

In one embodiment, the lighting system (100) achieves a PGDE Index in a range of 85% to 100%.

In one embodiment, the lighting system (100) reduces COVID-19 virus in the air stream by 99.9%.

In one embodiment, the lighting system (100) further comprises a motion sensor for protecting a person adjacent to or present within a predetermined distance of the lighting system from possible exposure to the ultraviolet light emitted by the ultraviolet light sources due to UVC light leakage.

In one embodiment, the motion sensor is a High Frequency Doppler (HFD) sensor.

In one embodiment, operating conditions and parameters of the ultraviolet light sources (107) and visible light sources (108) are adjusted and/or powered independently.

In one embodiment, luminous intensities, color temperatures, and on-off state of the ultraviolet light sources (107) and the visible light sources (108) are adjusted manually by input from a user, or are adjusted automatically based on instructions received from a smart home control appliance or encoded in software of the lighting system (100).

In one embodiment, the plurality of ultraviolet light sources (107) comprises two UVC LED strips facing one another, wherein air inside the lighting system flows in between the two UVC LED strips for the most potent irradiation and disinfection, wherein possible UVC leakage from the lighting system is minimized.

In one embodiment, the lighting system (100) further comprises an airflow detector included in the air circulation means (109) for detecting flow rate of air therein, wherein when the flow rate of air reaches a threshold value or a range, the airflow detector provides a signal to a driver of the lighting system (100) to adjust the power of the ultraviolet light sources (107).

In one embodiment, when the flow rate of air is lower (higher) than a predetermined value, the power of the ultraviolet light sources (107) is decreased (increased).

In one embodiment, the case frame (105) and the front cover assemble (101) are made of aluminum and have one or more reflecting surfaces for maximal reflection of UVC rays. In one embodiment, the surface characteristics can be found in WO 2017158989A1.

In one embodiment, the power of the ultraviolet light sources (107) is adjusted in a way to maintain a PGDE Index substantially in a range of 85% to 100%.

Throughout this application, various references or publications are cited. Disclosures of these references or publications in their entireties are hereby incorporated by reference into the application in order to more fully describe the state of the art to which this invention pertains. It is to be noted that the transitional term “comprising”, which is synonymous with “including”, “containing” or “characterized by”, is inclusive or open-ended, and does not exclude additional, un-recited elements or method steps.

### REFERENCES

1. Ultraviolet Disinfection Guidance Manual for the Final Long Term 2 Enhanced Surface Water Treatment Rule. EPA 815-R-06-007. United States Environmental Protection Agency (November 2006).
2. Murdoch et al., Bactericidal Effects of 405 nm Light Exposure Demonstrated by Inactivation of Escherichia, Salmonella, Shigella, Listeria, and Mycobacterium Species in Liquid Suspensions and on Exposed Surfaces. Scientific World J. Vol 2012, Article ID 137802 (2012).

What is claimed is:

1. A lighting system for disinfection of an air stream, comprising:
a front cover assembly comprising detachably embedded light transmissive members, a detachable inlet for receiving air, and a detachable outlet for exhausting air, wherein a stream of air is passable from the inlet to the outlet;
a casing frame including mounting openings;
a plurality of ultraviolet light sources detachably mounted on the mounting openings and configured to emit ultraviolet light with adjustable wavelengths and adjustable intensity to irradiate air in the interior of the lighting system;
a plurality of visible light sources detachably mounted on the mounting openings and configured to emit visible light with adjustable luminous intensity and/or color temperature, wherein the visible light transmits through the light transmissive members and propagates outside the lighting system; and
air circulation means detachably attached to the mounting openings and configured to move a stream of air through the inlet, from outside into the interior of the lighting system; within the lighting system, from the inlet towards the outlet; and, through the outlet, from the interior to outside the lighting system;
wherein the front cover assembly is detachably attached to the casing frame, such that the light transmissive members and the mounting openings are aligned; the front cover assembly and the casing frame jointly form the exterior of the lighting system as an enclosure protecting the ultraviolet light sources, the visible light sources, and the air circulation means; and, when the lighting system is operating, an air stream moved by the air circulation means enters the interior of the lighting system, is irradiated by the ultraviolet light sources, and exits the lighting system via the outlet, thereby disinfecting the air stream passing through the lighting system.

2. The lighting system of claim 1, wherein the ultraviolet light sources emit UVC rays with wavelength range of 200 to 280 nm.

3. The lighting system of claim 1, wherein the lighting system has a power of 30 to 150 W, a flux of 10 to 10,000 μW/cm² so as to achieve an irradiation dosage of at least 2,500 μW·s/cm² in the interior of the lighting system within several seconds to several minutes of use, and an irradiation of 200 to 3,000 mW for rapid and substantially complete disinfection of the air stream.

4. The lighting system of claim 1, further comprising an air filtration means attached to the air circulation means, facing the inlet and/or the outlet on the front cover assembly, and configured to remove particulates from the air stream.

5. The lighting system of claim 1, further comprising a UVC sensor inside the front cover assembly for detecting the intensity of UVC rays emitted by the ultraviolet light sources in the interior of the lighting system.

6. The lighting system of claim 5, wherein, based on sensory data received from the UVC sensor, a driver of the lighting system automatically modulates the electric power supplied to the ultraviolet light sources to maintain a constant level of UVC intensity in the interior of the lighting system.

7. The lighting system of claim 6, wherein, 70% of the nominal power supply is provided to brand-new ultraviolet light sources while, when the UVC sensor detects a 10% reduction of the intensity of UVC rays in the interior of the lighting system, 80% of their nominal power supply is provided to the ultraviolet light sources after use for a period time.

8. The lighting system of claim 6, wherein the ultraviolet light sources with modulable electric power have an increased longevity.

9. The lighting system of claim 8, wherein the ultraviolet light sources have a rated life of 30,000 to 50,000 hours, wherein the rated life is a period of time in which the ultraviolet light sources have an optical output of no less than 70% of the nominal power output.

10. The lighting system of claim 1, wherein the lighting system achieves a PGDE Index in a range of 85% to 100%.

11. The lighting system of claim 10, wherein the lighting system reduces COVID-19 virus in the air stream by 99.9%.

12. The lighting system of claim 1, further comprising a motion sensor for protecting a person adjacent to or present within a predetermined distance of the lighting system from possible exposure to the ultraviolet light emitted by the ultraviolet light sources due to UVC light leakage.

13. The lighting system of claim 12, wherein the motion sensor is a High Frequency Doppler (HFD) sensor.

14. The lighting system of claim 1, wherein operating conditions and parameters of the ultraviolet light sources and visible light sources are adjusted and/or powered independently.

15. The lighting system of claim 14, wherein luminous intensities, color temperatures, and on-off state of the ultraviolet light sources and the visible light sources are adjusted manually by input from a user, or are adjusted automatically based on instructions received from a smart home control appliance or encoded in software of the lighting system.

16. The lighting system of claim 1, wherein the plurality of ultraviolet light sources comprises two UVC LED strips facing one another, wherein air inside the lighting system flows in between the two UVC LED strips for the most potent irradiation and disinfection, wherein possible UVC leakage from the lighting system is minimized.

17. The lighting system of claim 1, further comprising an airflow detector included in the air circulation means for detecting flow rate of air therein, wherein when the flow rate of air reaches a threshold value or a range, the airflow detector provides a signal to a driver of the lighting system to adjust the power of the ultraviolet light sources.

18. The lighting system of claim 17, wherein, when the flow rate of air is lower than a predetermined value, the power of the ultraviolet light sources is decreased; and when the flow rate of air is or higher than a predetermined value, the power of the ultraviolet light sources is increased.

19. The lighting system of claim 1, wherein the case frame and the front cover assemble are made of aluminum and have one or more reflecting surfaces for maximal reflection of UVC rays.

20. The lighting system of claim 17, wherein the power of the ultraviolet light sources is adjusted in a way to maintain a PGDE Index substantially in a range of 85% to 100%.
